(19) United States
(12) Patent Application Publication
(10) Pub. No.: US 2006/0099223 A1
(43) Pub. Date: May 11, 2006

(54) Enzymetic hydrolysate of algae and method for preparing the same

(75) Inventors: Po-Han Chen, Tainan City (TW); Ming-Hung Huang, Xianxi Shiang (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Taiyen Biotech Co. Ltd

(21) Appl. No.: 11/041,692

(22) Filed: Jan. 24, 2005

(30) Foreign Application Priority Data
Nov. 11, 2004 (TW) 93134535

(51) Int. Cl. C12P 21/06; A61K 36/02

(52) U.S. Cl. 424/195.17; 435/68.1

(57) ABSTRACT

An enzymatic hydrolysate of algae and a method for preparing the same are disclosed. After rehydrating dry algae, an insoluble pellet is digested by using a specific enzymatic reaction condition, so as to produce the soluble and bioactive enzymatic hydrolysate of algae. The enzymatic hydrolysate of algae, which comprises a mixture of polysaccharides and polypeptides, can promote a growth of skin cells cultured in vitro and be formulated in cosmetic, food or skin external agent compositions.

### RELATED APPLICATIONS

The present application is based on, and claims priority from, Taiwan Application Serial Number 93134535, filed on Nov. 11, 2004, the disclosure of which is hereby incorporated by reference herein in its entirety.

### FIELD OF THE INVENTION

The present invention relates to a hydrolysate of algae and a method for preparing the same, and more particularly, to an enzymatic hydrolysate of algae by enzymatic treatment and a method for preparing the same.

### BACKGROUND OF THE INVENTION

Algae of the protista are at the base of the food web in the fresh water and ocean ecosystem and provide aquatic animals with energy and nutrient. The larger algae can act as food for human, and the smaller ones, such as Chlorella spp. of green algae, Spirulina spp. of the blue-green algae and the like, are also cultivated in mass and produced to the healthy food for human due to abundance in proteins, complete and balanced kinds of human essential amino acids, and richness in vitamins, poly-unsaturated fatty acids and trace elements.

Second, many researches show that lipopolysaccharides and phycocyanin contained in the Spirulina cells have the function of enhancing the animal bone marrow regeneration, the thymus and spleen growth, and serum protein biosynthesis, and immune system. Moreover, U.S. Pat. No. 5,585,365 discloses calcium spirulan (Ca-SP), purified from a hot water extract of Spirulina platensis, has the antiviral functions to treat herpes simplex virus, human immunodeficiency virus (HIV) and the like. In addition, many researches also indicate the algae extract having various effects, for example, alleviating diabetes and hypertension, reducing cholesterol, anticancer and the like.

Besides, the alga itself, its extract or hydrolysate is further added in cosmetics for providing various beauty effects. Examples of such can be found in TW 520,286, US Patent Application No. 2003/0091560, US Patent Application No. 2002/0160064, US Patent Application No. 2002/0120242, U.S. Pat. No. 6,190,664, U.S. Pat. No. 5,508,033, EU 1,239,813, RU 2,114,632, CN 1,206,587, FR 2,609,246, FR 2,555,444 and JP 52,021,336, and the above all is recited as references herein. The above patents disclose various cosmetic compositions containing Spirulina extract, such as freeze-dry Spirulina powder, algal extract of Chlorella and Spirulina, at least one isoflavone aglycone extracted from Spirulina, Spirulina extract from freeze-and-thaw cycles followed by NaCl solution extraction, Spirulina extract from hot water, and Spirulina hydrolysate extracted from organic solvents like ethanol/acetone and ethanol/water.

Accordingly, the prior skills of algae practices are not beyond the above description, for example, directly utilizing the algae, the algal extract from freeze-and-thaw cycles followed by NaCl solution extraction, the algal hydrolysate extracted from organic solvents or the algal extract from hot water. However, the alga itself is not easy to be absorbed by human body, and the bioactive substances contained in the algae are easily destroyed in the processes of freeze and thaw cycles, organic solvent or hot water extractions.

### SUMMARY OF THE INVENTION

Accordingly, it is an aspect of the present invention to provide an enzymatic hydrolysate of algae, which comprises a mixture of polysaccharides and polypeptides, capable of adding in a formulation of cosmetic, food or skin external medicine compositions.

It is another aspect of the present invention to provide a method for preparing enzymatic hydrolysate of algae, which uses a specific enzymatic condition acting on a insoluble pellet that is rehydrated from dry algae, so as to obtain the soluble and bioactive enzymatic hydrolysate of algae that comprises a mixture of polysaccharides and polypeptides.

According to the aforementioned aspect of the present invention, there is provided an enzymatic hydrolysate of algae, which is obtained by a insoluble pellet that is rehydrated from dry algal cells, such as the ones of Chlorella spp. or Spirulina spp., and then digested under a specific enzymatic condition for at least 1.5 hours, so as to obtain the soluble and bioactive enzymatic hydrolysate of algae, wherein the enzymatic hydrolysate of algae comprises a mixture of at least a polysaccharide and at least a polypeptide having a molecular weight ranging from 1000 daltons (Da) to 17000 Da.

Preferably, an enzyme used in the specific enzymatic condition may be an endopeptidase.

Preferably, the enzymatic hydrolysate of algae may be added in a formulation of cosmetic, food or skin external medicine compositions.

Besides, according to another aspect of the present invention, there is provided a method for preparing enzymatic hydrolysate of algae. Dry algal cells, such as the ones of Chlorella spp. or Spirulina spp., are rehydrated to obtain a soluble supernatant and an insoluble pellet. After removing the supernatant, the insoluble pellet is digested under a specific enzymatic condition for at least 1.5 hours, so as to obtain the soluble and bioactive enzymatic hydrolysate of algae, wherein the enzymatic hydrolysate of algae comprises a mixture of at least a polysaccharide and at least a polypeptide having a molecular weight ranging from 1000 Da to 17000 Da.

The enzymatic hydrolysate of algae is obtained by using the method of the present invention, which uses the specific enzymatic condition acting on a insoluble pellet that is rehydrated from dry algae, so as to obtain the soluble and bioactive enzymatic hydrolysate of algae. The enzymatic hydrolysate of algae comprises a mixture of polysaccharides and polypeptides, capable of adding in a formulation of cosmetic, food or skin external medicine compositions.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows the histogram of the relative cell viability of skin cells treated with the enzymatic hydrolysates of algae according to the preferred embodiments of the present invention.

### DETAILED DESCRIPTION OF THE PREFERRED EMBODIMENT

The present invention provides an enzymatic hydrolysate of algae and a method for preparing the same, which uses a specific enzymatic condition acting on a insoluble pellet that is rehydrated from dry algae, so as to obtain the soluble and bioactive enzymatic hydrolysate of algae.

As used herein, the term “Chlorella spp.” or “Spirulina spp.” generally refers to any species of algae belonging to the genus Chlorella or Spirulina, respectively.

As used herein, the term “algal cells” generally refers to living, wet, or dry algal cells, and preferably, dry algal cells act as a starting material.

Dry algal cells such as the ones of Chlorella spp. or Spirulina spp., are rehydrated in distilled, deionized, other purified water or a buffered solution at room temperature, so as to obtain an algal suspension that has any concentration depending on the requirement. In an exemplary embodiment, the concentration of the algal suspension includes but not is not intended to be limited to, for example, 20% to 40% by weight, wherein the algal suspension contains a soluble supernatant and an insoluble pellet. After removing the soluble portion by centrifugation or filtration, the insoluble pellet is digested under a specific enzymatic condition for at least 1.5 hours, so as to obtain the soluble and bioactive enzymatic hydrolysate of algae.

In a preferred embodiment of the invention, an appropriate enzyme used in the specific enzymatic condition as described above may be an endopeptidase, and preferably, pepsin, trypsin, papain or other proteases. In an example, the insoluble pellet is digested by 0.15% to 10% of the pepsin by weight at a temperature ranging from 25 Celsius degrees to 55 Celsius degrees and under pH 1 to pH 5. In another example, the insoluble pellet is digested by 0.15% to 10% of the trypsin by weight at a temperature ranging from 25 Celsius degrees to 50 Celsius degrees and under pH 5 to pH 9. In a further example, the insoluble pellet is digested by 0.15% to 10% of the papain by weight at a temperature ranging from 25 Celsius degrees to 75 Celsius degrees and under pH 5 to pH 10.

After the above endopeptidase treatment, the resultant enzymatic hydrolysate of algae comprises a mixture of at least a polysaccharide and at least a polypeptide having a molecular weight ranging from 1000 Da to 17000 Da, capable of effectively enhancing a growth of skin cells cultured in vitro and adding in a formulation of cosmetic, food or skin external medicine compositions.

Hereinafter, the enzymatic hydrolysate of algae and a method for preparing the same of the present invention are more explicitly clarified in following preferred embodiments. However, the embodiments are merely given to illustrate various applications of the invention rather than to be interpreted as limiting the scope of the appended claims.

### EXAMPLE 1

The dry Spirulina cells are rehydrated to a Spirulina suspension at, for example, 28% by weight containing a supernatant and an insoluble pellet. After removing the supernatant, the insoluble pellet is resuspended in a buffered solution of about pH 2.5, and digested by, for example, 0.57% of pepsin by weight at room temperature (about Celsius degrees). The enzymatic reaction is stopped after digesting for 0.5 hour, 1.0 hour, 1.5 hours, 2.0 hours and 16.0 hours, respectively, so as to obtain the enzymatic hydrolysate of algae.

### EXAMPLE 2

The dry Spirulina cells are rehydrated to a Spirulina suspension of, for example, % by weight containing a supernatant and an insoluble pellet. After removing the supernatant, the insoluble pellet is resuspended in a buffered solution of about pH 3.5, and digested by, for example, 0.57% of pepsin by weight at room temperature (about 25 Celsius degrees). The enzymatic reaction is stopped after digesting for 0.5 hour, 1.0 hour, 1.5 hours, 2.0 hours and 16.0 hours, respectively, so as to obtain the enzymatic hydrolysate of algae.

### EXAMPLE 3

The dry Spirulina cells are rehydrated to a Spirulina suspension at, for example, 30% by weight containing a supernatant and an insoluble pellet. After removing the supernatant, the insoluble pellet is resuspended in a buffered solution of about pH 6.5 to about pH 7.5, and digested by, for example, 0.57% of papain by weight at room temperature (about 25 Celsius degrees). The enzymatic reaction is stopped after digesting for 0.5 hour, 1.0 hour, 1.5 hours, 2.0 hours and 16.0 hours, respectively, so as to obtain the enzymatic hydrolysate of algae.

### EXAMPLE 4

The dry Spirulina cells are rehydrated to a Spirulina suspension at, for example, 35% by weight containing a supernatant and an insoluble pellet. After removing the supernatant, the insoluble pellet is resuspended in a buffered solution of about pH 6.5 to about pH 7.5, and digested by, for example, 1% of papain by weight at about 50 Celsius degrees. The enzymatic reaction is stopped after digesting for 0.5 hour, 1.0 hour, 1.5 hours, 2.0 hours and 16.0 hours, respectively, so as to obtain the enzymatic hydrolysate of algae.

### EXAMPLE 5

The dry Spirulina cells are rehydrated to a Spirulina suspension at, for example, 25% by weight containing a supernatant and an insoluble pellet. After removing the supernatant, the insoluble pellet is resuspended in a buffered solution of about pH 6.5 to about pH 7.5, and digested by, for example, 0.57% of trypsin by weight at room temperature (about 25 Celsius degrees). The enzymatic reaction is stopped after digesting for 0.5 hour, 1.0 hour, 1.5 hours, 2.0 hours and 16.0 hours, respectively, so as to obtain the enzymatic hydrolysate of algae.

### EXAMPLE 6

The dry Spirulina cells are rehydrated to a Spirulina suspension at, for example, 20% by weight containing a supernatant and an insoluble pellet. After removing the supernatant, the insoluble pellet is resuspended in a buffered solution of about pH 9.0, and digested by, for example, 0.25% of trypsin by weight at room temperature (about 25 Celsius degrees). The enzymatic reaction is stopped after digesting for 0.5 hour, 1.0 hour, 1.5 hours, 2.0 hours and 16.0 hours, respectively, so as to obtain the enzymatic hydrolysate of algae.

### EXAMPLE 7

The enzymatic hydrolysates of algae obtained from Examples 1 to 6 are analyzed by a typical sodium dodecyl sulfate-polyacrylamide gel electrophoresis (SDS-PAGE). The resultant polypeptides are determined in the molecular weights ranging from 1000 Da to 17000 Da (not shown). Next, the enzymatic hydrolysates of algae obtained from Examples 1 to 6 are subject to a growth test of skin cells cultured in vitro. Typically, mammalian fibroblasts act as the skin cells. In the case of Example 7, the rodent 3T3 fibroblasts are cultured in a 96-well culture plates at a cell density of about 1×10⁵ cells for each well. The 3 T3 fibroblasts are cultured in the cell medium added with 1% to 10% of the enzymatic hydrolysates of algae by weight obtained from Examples 1 to 6 at 37 Celsius degrees in an atmosphere of humidified 5% CO₂ for 48 hours. At the end of each incubation period, the cell viability is quantitatively analyzed by 3-[4,5-Dimethylthylthiazol-2-yl]-2,5-Diphenyltetrazolium Bromide (MTT) assay. Reference is made to TAB. 1, which shows the relative cell viability treated with the enzymatic hydrolysates of algae according to the preferred embodiments of the present invention, wherein each cell viability is normalized to the control cell viability that is unexposed to any enzymatic hydrolysates of algae as 100%.

TABLE 1

| Incubation | EXAMPLE | | | | | |
|---|---|---|---|---|---|---|
| Time (Hour) | 1 | 2 | 3 | 4 | 5 | 6 |
| | Cell Viability (%) | | | | | |
| 0.5 | 103.76 | 104.03 | 96.97 | 94.79 | 95.90 | 104.21 |
| 1.0 | 97.86 | 97.94 | 97.53 | 95.38 | 122.17 | 135.43 |
| 1.5 | 149.90 | 148.61 | 155.82 | 148.40 | 136.99 | 136.30 |
| 2.0 | 140.28 | 133.50 | 140.17 | 137.36 | 148.16 | 138.23 |
| 16.0 | 138.48 | 134.79 | 157.51 | 134.13 | 153.14 | 148.60 |

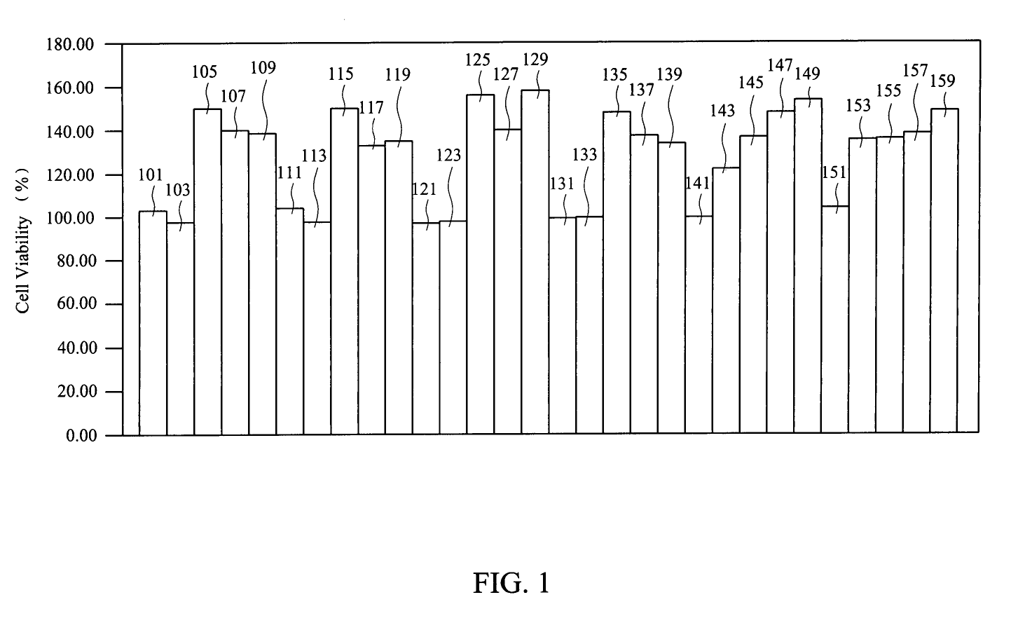

The result of TAB. 1 is further illustrated with FIG. 1. Reference is made to FIG. 1, which shows the histogram of the relative cell viability of skin cells treated with the enzymatic hydrolysates of algae according to the preferred embodiments of the present invention, wherein the vertical axis refers to the cell viability (%) with respect to the control cells, and the horizontal axis refers to the enzymatic hydrolysates of algae obtained by EXAMPLES 1 to 6 that are treated in various time from left to right columns. The columns 101, 103, 105, 107 and 109 show the cell viabilities treated with the enzymatic hydrolysates of algae in 0.5, 1.0, 1.5, 2.0 and 16 hours according to EXAMPLE 1, respectively. The columns 111, 113, 115, 117 and 119 show the cell viabilities treated with the enzymatic hydrolysates of algae in 0.5, 1.0, 1.5, 2.0 and 16 hours according to EXAMPLE 2, respectively. The columns 121, 123, 125, 127 and 129 show the cell viabilities treated with the enzymatic hydrolysates of algae in 0.5, 1.0, 1.5, 2.0 and 16 hours according to EXAMPLE 3, respectively. The columns 131, 133, 135, 137 and 139 show the cell viabilities treated with the enzymatic hydrolysates of algae in 0.5, 1.0, 1.5, 2.0 and 16 hours according to EXAMPLE 4, respectively. The columns 141, 143, 145, 147 and 149 show the cell viabilities treated with the enzymatic hydrolysates of algae in 0.5, 1.0, 1.5, 2.0 and 16 hours according to EXAMPLE 5, respectively. The columns 151, 153, 155, 157 and 159 show the cell viabilities treated with the enzymatic hydrolysates of algae in 0.5, 1.0, 1.5, 2.0 and 16 hours according to EXAMPLE 6, respectively.

As shown in TAB. 1 and FIG. 1, generally, the enzymatic hydrolysates of algae treated with pepsin, trypsin or papain in at least 1.5 hours can effectively enhance the growth of 3T3 fibroblasts cultured in vitro. The cell viability treated with the enzymatic hydrolysates of algae is about 30% to about 60% more than the cell viability of the control cells, wherein the enzymatic hydrolysates of algae even treated with trypsin in 1.0 hour can enhance the growth of 3T3 fibroblasts cultured in vitro.

In brief, the present invention utilizes a specific enzymatic condition acting on a insoluble pellet that is rehydrated from dry algae cells, so as to obtain the soluble and bioactive enzymatic hydrolysate of algae. It is worth mentioning that the enzymatic hydrolysate of algae produced by the present invention can effectively enhance a growth of skin cells cultured in vitro, and be further added to a formulation of cosmetic, food or skin external medicine compositions. In the case of the cosmetic composition, the enzymatic hydrolysate of algae of the present invention can added in a base at 0.1% to 50% by weight, and the base may be a cream, lotion, essence, gel or the like.

According to the aforementioned preferred embodiments, one advantage of the enzymatic hydrolysate of algae of the present invention comprises a mixture of polysaccharides and polypeptides, capable of effectively enhancing the growth of skin cells cultured in vitro and adding in the formulation of cosmetic, food or skin external medicine compositions.

According to the aforementioned preferred embodiments, another advantage of the method for preparing enzymatic hydrolysate of algae utilizes a specific enzymatic condition acting on a insoluble pellet that is rehydrated from dry algae, so as to obtain the soluble and bioactive enzymatic hydrolysate of algae. The enzymatic hydrolysate of algae comprises a mixture of polysaccharides and polypeptides, capable of effectively enhancing the growth of skin cells cultured in vitro.

As is understood by a person skilled in the art, the foregoing preferred embodiments of the present invention are illustrated of the present invention rather than limiting of the present invention. It is intended to cover various modifications and similar arrangements included within the spirit and scope of the appended claims. Therefore, the scope of which should be accorded the broadest interpretation so as to encompass all such modifications and similar structure.

1. An enzymatic hydrolysate of algae obtained by an insoluble pellet that is rehydrated from dry algae and digested by an endopeptidase for at least 1.5 hours, so as to obtain the soluble and bioactive enzymatic hydrolysate of algae, wherein the desiccated algae are selected from the group consisting of Chlorella spp. and Spirulina spp., and the enzymatic hydrolysate of algae comprises a mixture of at least a polysaccharide and at least a polypeptide having a molecular weight ranging from 1000 daltons (Da) to 17000 Da.

2. The enzymatic hydrolysate of algae according to claim 1, wherein the endopeptidase is selected from the group consisting of pepsin, trypsin, papain and other proteases.

3. The enzymatic hydrolysate of algae according to claim 2, wherein the insoluble pellet is digested by the pepsin at a temperature ranging from 25 Celsius degrees to 55 Celsius degrees and under pH 1 to pH 5.

4. The enzymatic hydrolysate of algae according to claim 2, wherein the insoluble pellet is digested by the trypsin at a temperature ranging from 25 Celsius degrees to 50 Celsius degrees and under pH 5 to pH 9.

5. The enzymatic hydrolysate of algae according to claim 2, wherein the insoluble pellet is digested by the papain at a temperature ranging from 25 Celsius degrees to 75 Celsius degrees and under pH 5 to pH 10.

6. The enzymatic hydrolysate of algae according to claim 1, wherein the ezymatic hydrolysate of algae is an additive of a cosmetic composition, an active component of a skin external medicine composition or a food additive.

7. The enzymatic hydrolysate of algae according to claim 6, an amount of the enzymatic hydrolysate of algae added in a base is from 0.1% by weight (wt. %) to 50 wt. % and the base is selected from the group consisting of a cream, a lotion, an essence and a gel.

8.-12. (canceled)
